(19) United States
(12) Patent Application Publication
(10) Pub. No.: US 2002/0169386 A1
(43) Pub. Date: Nov. 14, 2002

(54) Real-time biological-chemical-physical sensitivity and irritation onset neuro sensory (BIOCAPSIONS) biofeedback monitor

(76) Inventor: Robert L. Johnson JR., Adamsville, AL (US)

Correspondence Address: Robert L. Johnson, Jr., 1227 Shady Grove Road, Adamsville, AL, 35005, US

(21) Appl. No.: 10/053,046

(22) Filed: Nov. 10, 2001

Related U.S. Application Data
(60) Provisional application No. 60/247,250, filed on Nov. 13, 2000.

(51) Int. Cl. A61B 5/00

(52) U.S. Cl. 600/544; 600/300; 128/903; 128/905; 600/546

(57) ABSTRACT

An artificially intelligent dual real-time monitoring and measurement device that includes stimulating, recording, transdermal pharmacological delivery electrodes, a differential amplifier, A/D converter, digital filtering, digital signal processor and software for measurement, display and storage of analog data in a personal digital computer monitoring device; the portable biofeedback monitor, can accurately monitor the onset of chemical and/or physical irritation and has visual, audible and vibratory alerts, windows compatible menu driven software, 25 biofeedback displays including bar graphs, power spectra analysis, wave form display and multi-color strobe lights. The most novel attribute of the RT/BIOCAPSIONS biofeedback monitor is that it can be used as a tool to counter terrorism. My invention represents a positive solution to alleviate panic over the threat of biological-chemical-physical terrorism while at the same time offering a mass early medical surveillance & intervention strategy to address the urgent national need for biochemical counter-terrorism strategies. This invention performs determinations and displays expressions of Human BIOCAPSIONS receptive fields and biosensors, analog feature node expression, pattern constant processing, pattern-directed retrieval, connectivity, matching, recognition, variability, classification, pattern-directed modulation, pattern-directed interfacing, stimulus-equivalence reconciliation, bio-feature detection and association and any and all embodiments within the scope of the invention.

### CROSS REFERENCE TO RELATED DISCLOSURE DOCUMENT AND PROVISIONAL PATENT APPLICATION

This invention was disclosed in Information Disclosure Document filed with the United States Patent and Trademark Office on Aug. 13, 1991, and Provisional Patent Application filed with the United States Patent and Trademark Office on Nov. 13, 2000.

### BACKGROUND OF THE INVENTION

1. Field of Invention

This invention relates to an artificially intelligent real-time biological-chemical and physical sensitivity and irritation onset neuro sensory (RT/BIOCAPSIONS) biofeedback monitor that also performs real time monitoring of radiation injury onset (REMRIO). More specifically, it relates to a dual monitoring and measurement device that includes two recording, a stimulating, and a transdermal pharmacological delivery electrodes, differential amplifier, A/D converter, digital filtering, digital signal processor and software for measurement, display and storage of analog data in a personal digital computer measurement device that is compact, portable and field durable for military use and for applications radiation safety, sun or uv lamp skin tanning, allergy and multiple chemical sensitivity testing, and industrial hygiene monitoring and bioassay procedures.

2. Description of Prior Art

1. On-Line Abstract Compilation: Oct. 13, 2001, Related Works at the National Academy of Sciences, (http://www4.nas.edu/PGA/rap.nsf/ByTitle/

a) RO# 97.30.00.B3824: “Soldier Physiological Status Monitoring,” US Army Medical Research and Materiel Command, US Army Research Institute of Environmental Medicine;

b) RO# 76.31.09.B3252: “Biological Recognition Systems for Use in Biosensors,” US Army Research Laboratory, Sensors and Electron Devices Directorate;

c) RO# 56.56.01.B2550: “Physiological Monitoring of Alertness and Attention,” Naval Health Research Ctr./Naval Health Research Ctr., Behavioral Psychology;

d) RO# 44.10.03.B4693: “Neuroelectric Signal Processing for Control and Human Performance,” NASA Ames Research Center, Engineering and Applied Sciences;

e) RO# 11.01.05.B4467: “Infrared Spectrometry: Application to Environmental Sensing,” US Army Edgewood Chemical and Biological Center-US Army Soldier and Biological Chemical Command, Mathematics;

f) RO# 97.25.05.B3493: “Neurotoxicology: Electrophysiology,” US Army Medical Research and Materiel Command, US Army Medical Research Institute of Chemical Defense, Pharmacology Division;

g) RO# AA.33.01.B4683: “Photonic Analog-to-Digital Conversion,” US Military Academy, Photonics Research Laboratory;

h) RO# 11.01.04.B0041: “Electrophysiological Effects of Chemical Agents,” US Army Edgewood Chemical and Biological Center-US Army Soldier and Biological Chemical Command, Life Sciences, Medical Sciences and Behavioral Sciences;

i) RO# 97.25.05.B3494: “Neurotoxicology: Electrophysiology and Neuropharmacology,” US Army Medical Research and Materiel Command, US Army Medical Research Institute of Chemical Defense, Pharmacology Division;

j) RO# 13.15.10.B3742: “Human Thermophysiological Responses to Nonionizing Radiation,” US Air Force Research Laboratory, Human Effectiveness Directorate;

2. On-Line Abstract, “Autonomic Nervous System and Smooth Muscle responses to Combined Insult: Ionizing Radiation plus Enteropathogens or Biological Warfare Agents and Their Treatments,” Armed Forces Radiobiology Research Institute, Nov. 28, 2000.

3. Provisional Patent Application, “Real-Time Chemical and Physical Sensitivity and Irritation Onset Neuro Sensory Biofeedback Monitor” U.S. PTO Appl# 60/247,250 Nov. 13, 2000.

4. Abstract Disclosure and Presentation, “An In Vitro Model For Assessing The Small Intestinal Sensory Response To Ultraviolet Radiation”, Argonne National Laboratory Public Symposium, November 1990.

5. NeuroTan Monitor PTO Disclosure Document, Aug. 13, 1991.

### Prior Art Build and Test

6. Invention Submission Corporation Disclosure, March 1992.

7. Metropolitan Center for High Technology, November 1992, business incubator program counseling.

8. SBIR Phase I Grant Application, May 1993; human systems/subsystems, behavioral chronopharmacological technology, automatic system for detection of microwave related personnel hazards.

9. U.S. Air Force Armstrong Laboratory, June 1993, unsolicited proposal focal point.

10. Argonne National Laboratory, June 1993, proposed CRADA agreement for radiobiology and software development.

### Prior Art Referenced

11. U.S. Pat. No. 5,413,113, issue May 9, 1995 to Robert Milne describes an electronic allergo-sensivity test device.

12. U.S. Pat. No. 4,702,259, issued Oct. 27, 1987 to Marc Ferreira et al., discloses a device for measuring and indicating changes in the electrical resistance of a living body.

13. U.S. Pat. No. 4,805,621 issued to Roland Heinze et al. on Feb. 21, 1989 discloses an apparatus for measuring the impedance of body tissue with a signal source connected to the tissue to be measured which supplies an electrical signal to the tissue.

14. U.S. Pat. No. 4,809,707 issued to Thomas L. Kraft et al. on Mar. 7, 1989 discloses an electrode for performing transdermal delivery.

15. U.S. Pat. No. 4,819,657, issued on Apr. 11, 1989, also to Thomas L. Kraft discloses an automatic allergy detection system.

### Major Improvements Over Prior Art Real-Time NeuroTan Monitor, 1991, 1993;

(1) Electromagnetic Shielding

(2) Fiber Optic Recording Electrodes

(3) Reduced Size and Weight

(4) Increased Data Processing Speed

(5) Enhanced Device Analog-to-Digital Conversion

(6) Impulse Pattern Recognition Processor

(7) Enhanced Device Input Keypad and Function Menus

(8) Enhanced Device Output Alerts, Reports, Graphics

(9) Special Application in Chemical Sensitivity Neuromonitoring

(10) Special Application in Industrial Hygiene Neuromonitoring

(11) Enhanced Pharmacological Transdermal Delivery Systems

### Major Improvements Over Prior Art ELAST Device, 1993, 1994, 1995

(1) Fourth Electrode

(2) Transdermal Delivery System for Advanced Military Personnel Protection

(3) Compact, Portability and Field Durable for Military Use

(4) Physical Sensitivity Monitoring and Measurement Capability

### Major Improvements Over Prior Art Real-Time CAPSIONS, 2000

(1) Enhanced ability to detect sensitivity and irritation onset resulting from exposure to noxious stimuli of biological etiology

### Major Limitations of ELAST Device, 1995

(1) Lacks Base-Line Stimulus Evoked Potential Response

(2) Fixed Location, Non Field-Portable Usage Design

(3) Lacks Durability for Occupational, Military and Consumer Use

(3) Not Multi-Modal; Limited to Galvanic Skin Measurement

### SUMMARY OF THE INVENTION

### Ramifications

This artificially intelligent RT/BIOCAPSIONS biofeedback monitor represents a positive solution to alleviate panic over the threat of biological and chemical terrorism while at the same time offering a mass early medical surveillance & intervention strategy to address the urgent national need for biochemical counter-terrorism strategies.

### Scope

This artificially intelligent RT/BIOCAPSIONS biofeedback monitor is a device that provides real-time measurement of exposure to various types of biological and chemical and physical stimuli while providing early warning against over-exposure to high and low level chemicals suspect of causing biological-chemical-physical hypersensitivity values based on data representative but not limited to human receptor potentials, electronic potentials, impulse discharge coding, periods and frequency, signaling and propagation, specificity, intensity, gradation, adaptation, amplification, transduction plasticity, convergence, divergence, and neural circuits and pathways. The continuous measurement protocol approach utilizes impulse pattern recognition algorithms and software to look for likely and unlikely groupings of neuronal responses when variable intensity biological and/or chemical and/or physical sensitizers are used as a neurological stimulus. The specific device processor routines characterize the types and ways in which sensory neurones respond to low level biological and physical and multiple chemical stimuli utilizing software conversion of said analog signals to time segments, applying said time segments to a high-pass filter, using said computer processor to determine the amplitude of said filter's digital output, and comparing said amplitude to a predetermined neuro-sensory pattern constant database and then generate device display output.

### Application

In accordance with the present invention a dual monitoring/measurement device equipped with behavioral chronopharmacological transdermal delivery system with applications in advanced military personnel protection, clinical treatment, industrial hygiene, skin tanning; as well as asthma, multiple chemical sensitivity, allergy prevention. The RT/BIOCAPSIONS biofeedback monitor represents a positive solution to alleviate panic over the threat of biological and chemical terrorism while at the same time offering a mass early medical surveillance & intervention strategy to address the urgent national need for biological and chemical and radiological counter-terrorism strategies.

### Method

A method using a computer processor to analyze neuronal-based electrical signals and data representative of human neuro-sensory activity in response to variable intensity biological and chemical and physical stimulation by converting said analog signals to time segments, applying said time segments to a high-pass filter, using said computer processor to determine the amplitude of said filter's digital output, comparing said amplitude to a predetermined neuro-sensory device display values for everyday living environment and occupational health time weighted averages, threshold limit values, and bioassays, and equipped with pharmacological transdermal delivery systems.

### Novelty

The novelty of my invention is that it is a portable, personal biofeedback monitor. Unlike the NeuroTan Monitor; the current invention is capable of displaying biological, chemical or physical sensitivities. My invention is a novelty over the ELAST device in that it has the capability to measure physical sensitivities as well as secondary physiological states. Another unobvious feature of my invention is the ability measure multi-modal neurosensory pathways in terms of industrial hygiene bioassays.

The most novel attribute of this artificially intelligent RT/BIOCAPSIONS biofeedback monitor is that it can be used as a tool to counter terrorism. My invention represents a positive solution to alleviate panic over the threat of biological-chemical-physical terrorism while at the same time offering a mass early medical surveillance & intervention strategy to address the urgent national need for biological and chemical and radiological counter-terrorism strategies.

The novelty of my invention in relation to military and civil defense support, is that this low-powered, miniaturized and field portable measurement device can be used to timely capture and distribute clinical information using network technology by utilizing the device's diagnostic capability, continuous data acquisition, non-invasive sensing of vital signs and body chemistry, immediacy of analysis prior to injury or illness, and ability to transmit this information to a distance monitoring facility to support decisions to plan and manage overall responses including transdermal pharmacological intervention.

### BRIEF DESCRIPTION OF DRAWINGS

Various other objects, features, and attendant advantages of the present invention will become more fully appreciated as the same becomes better understood when considered in conjunction with the accompanying drawings, in which like reference characters designate the same or similar parts throughout the several views, and wherein. The drawings in relation to my invention are:

FIG. 1 show general flow chart.

FIG. 2 show analog smoothing and monitor device output flow chart.

FIG. 3 show digital processing and measurement device output flow chart.

FIG. 4 show internal memory and external programming read/write interface flow chart.

FIG. 5 show external views.

FIG. 6 show internal views.

FIG. 7 show side views.

FIG. 8 show electrodes.

### DETAILED DESCRIPTION OF THE PREFERRED EMBODIMENTS

The following is a list of the elements discussed in the above specification.

Reference Numerals in Drawings

| | |
|---|---|
| 10 | On/Off Switch |
| 12 | Lighted Display Screen |
| 14 | On Screen Menu Selections |
| 16 | Function Keys |
| 18 | Numerical Key Pad |
| 20 | Alpha Key Pad |
| 22 | Preset User Parameter Buttons |
| 24 | Visual Alert Lights |
| 26 | Audio Alert Speaker |
| 30 | Screw Fasteners |
| 32 | Belt Clip |
| 52 | Battery Holder |
| 54 | Electrical Stimulator |
| 56 | Recording Electrode Jack |
| 58 | External Interface Jack |
| 60 | Vibratory Alert |
| 62 | External Interface Program Memory |
| 70 | Internal Program Memory |
| 72 | Main Internal Storage Memory |
| 74 | Smoothing Filter and Meter |
| 76 | Rectifier |
| 78 | Band Filters |
| 80 | Differential Amplifier |
| 82 | Electrical Stimulator |
| 84 | Electrode Bus |
| 86 | Integrator |
| 88 | Clock |
| 90 | Case Cover Attachments |
| 100 | Transdermal Delivery Electrode |
| 102 | Electrode Wire |
| 104 | Electrode Harness Plug-in |

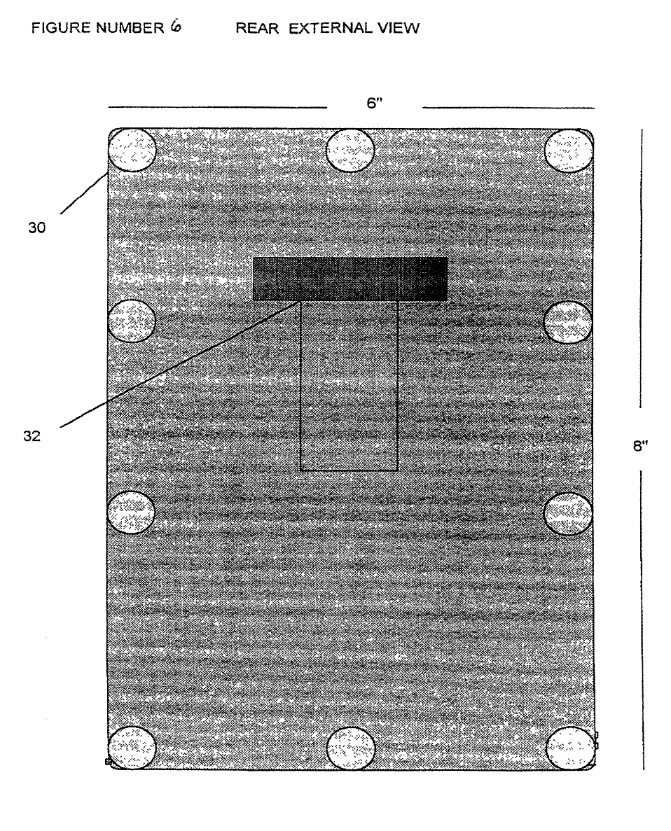

### Description of Preferred Embodiments—FIGS. 5 to 7

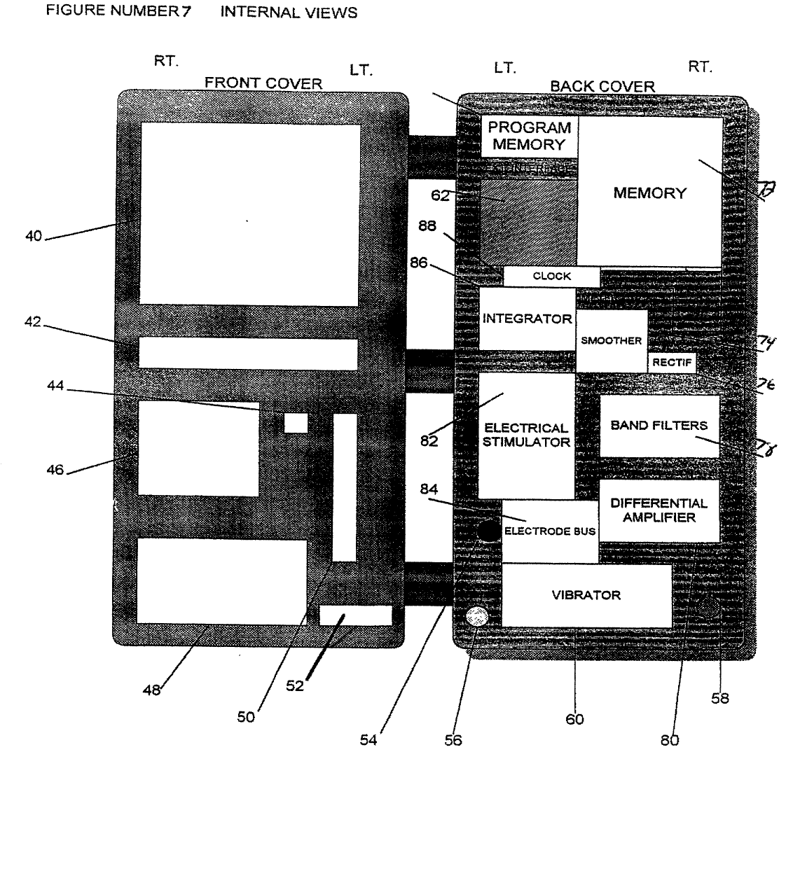

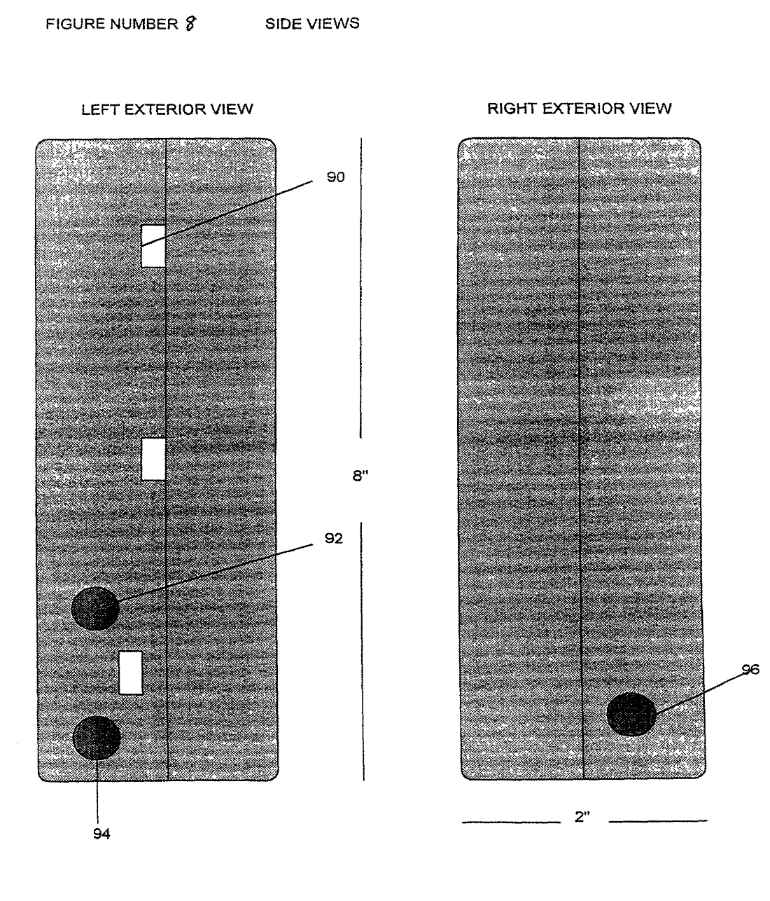

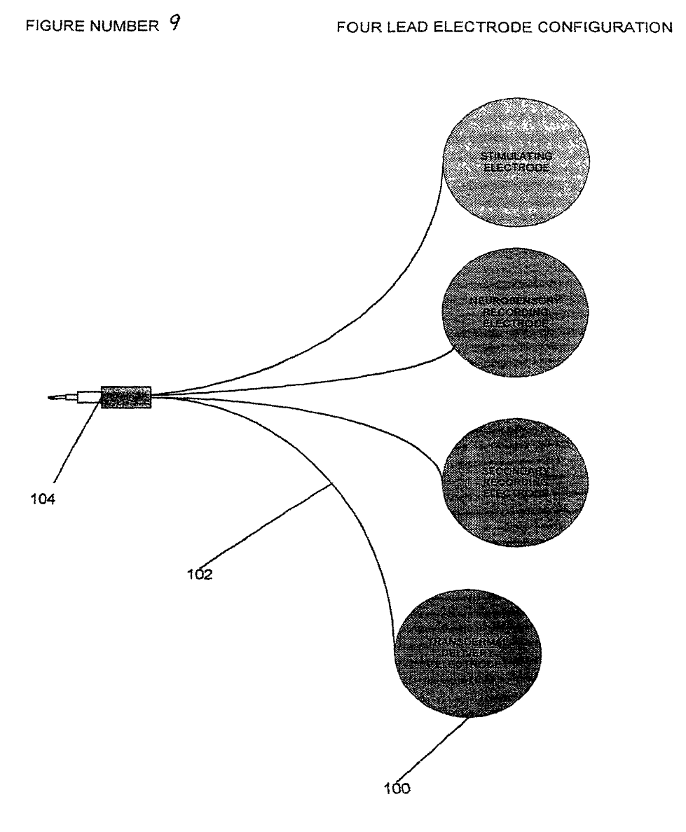

A typical embodiment of a REMRIO/CAPSIONS invention is illustrated in FIG. 5 (front exterior view), FIG. 5B (rear external view), FIG. 6 (internal views), FIG. 7 (exterior side views, and FIG. 7B (electrode views). There are various possibilities with regard to the relative disposition of the user input pad, display screen, audio alerts, vibratory alerts, visual alerts, size and weight, electrodes, microprocessors, casing materials. Adaptation for military requirements further increase the possibilities.

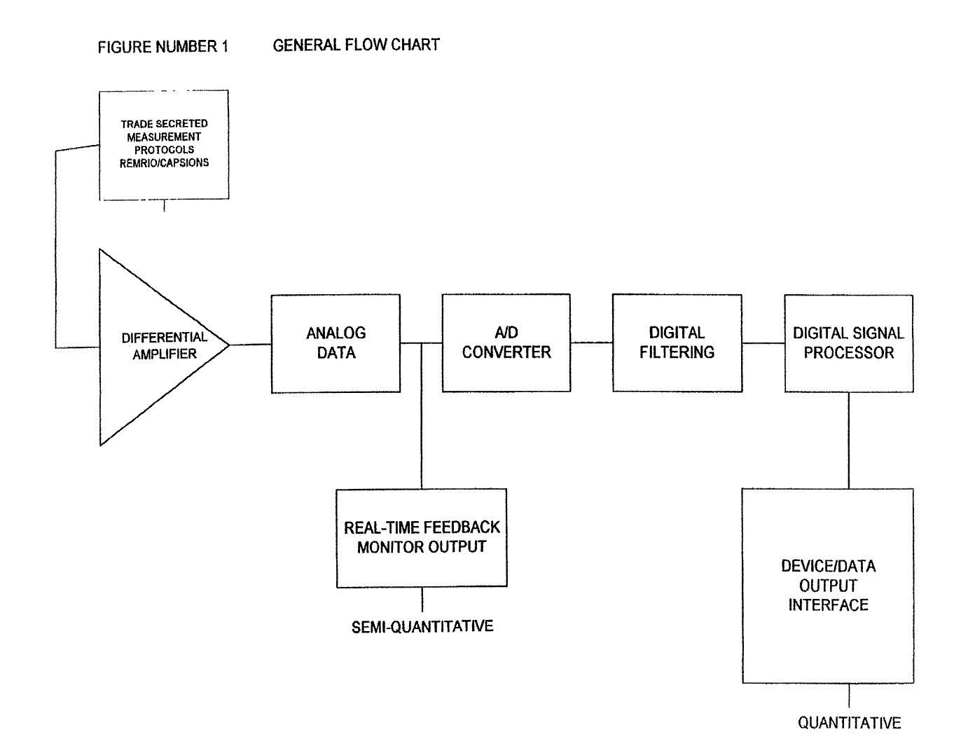

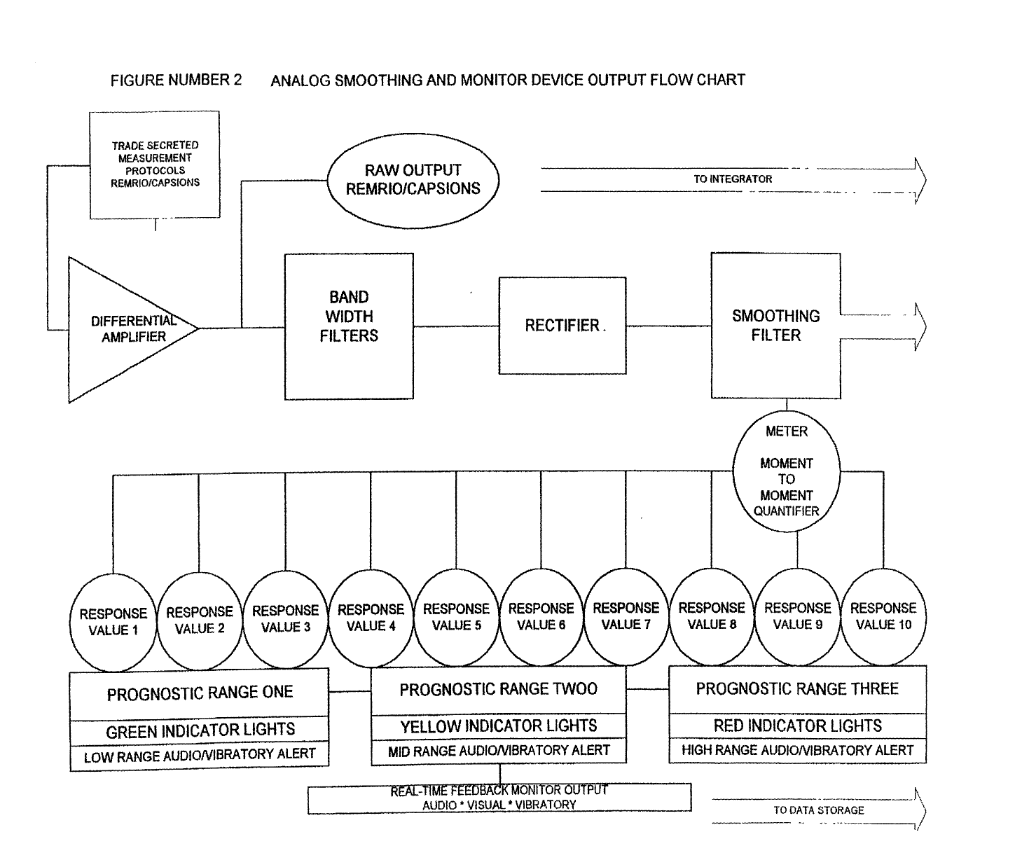

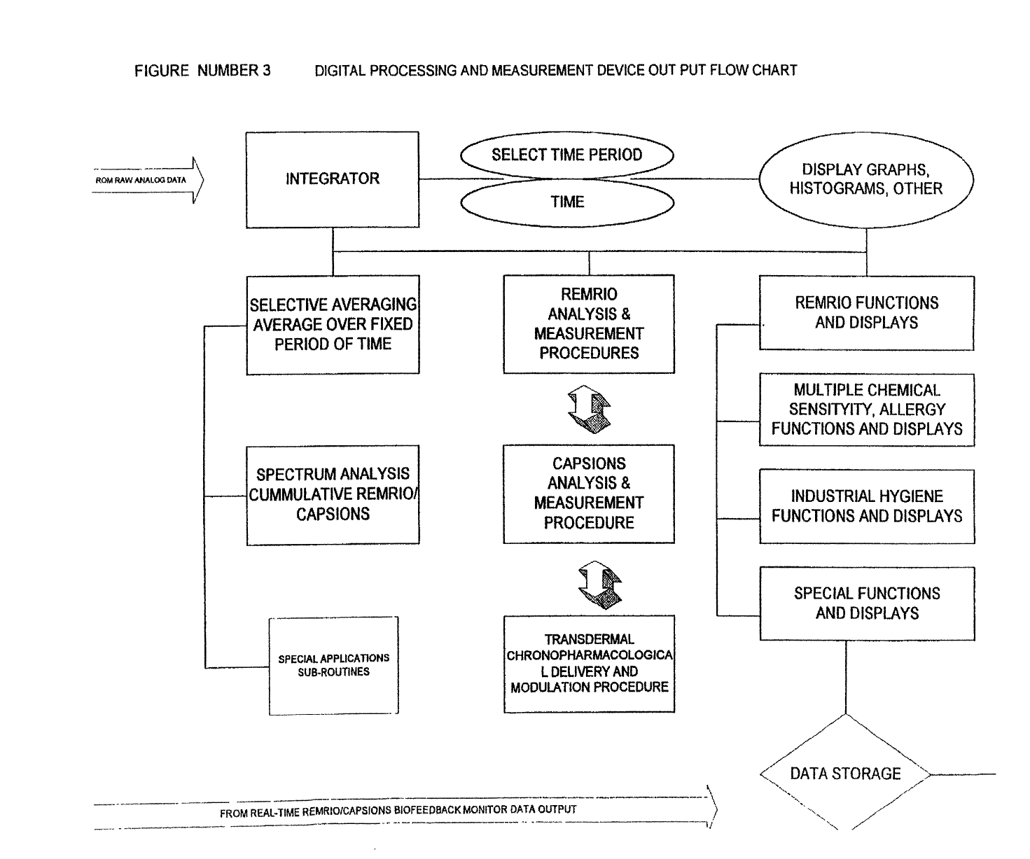

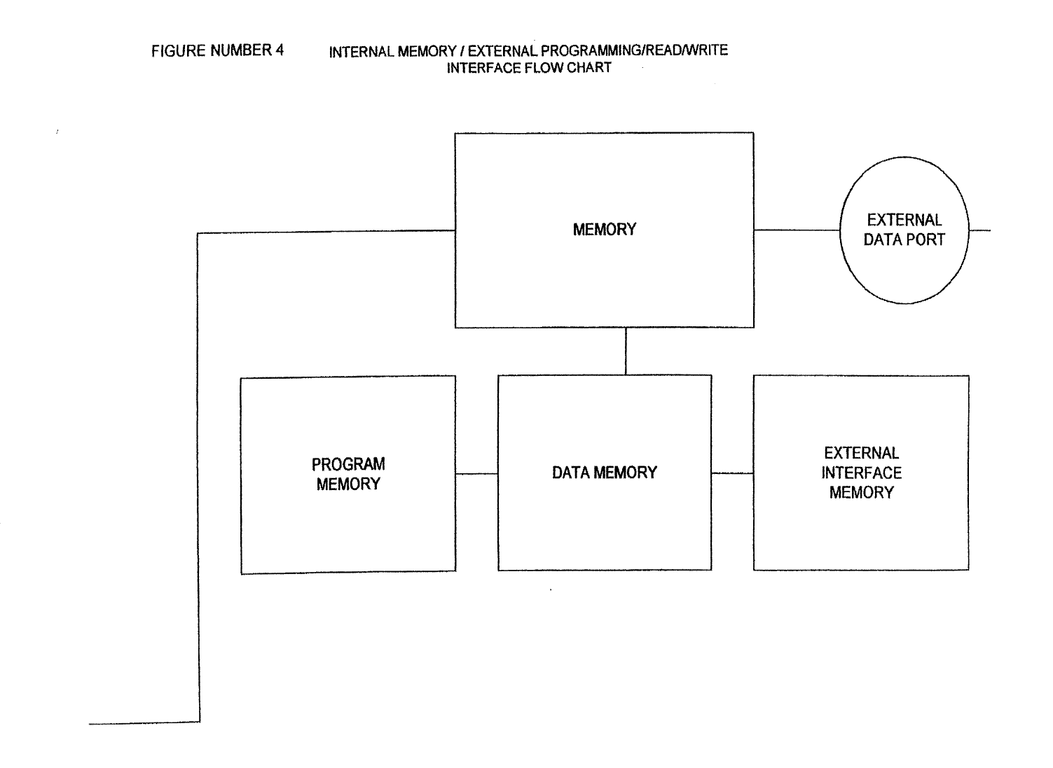

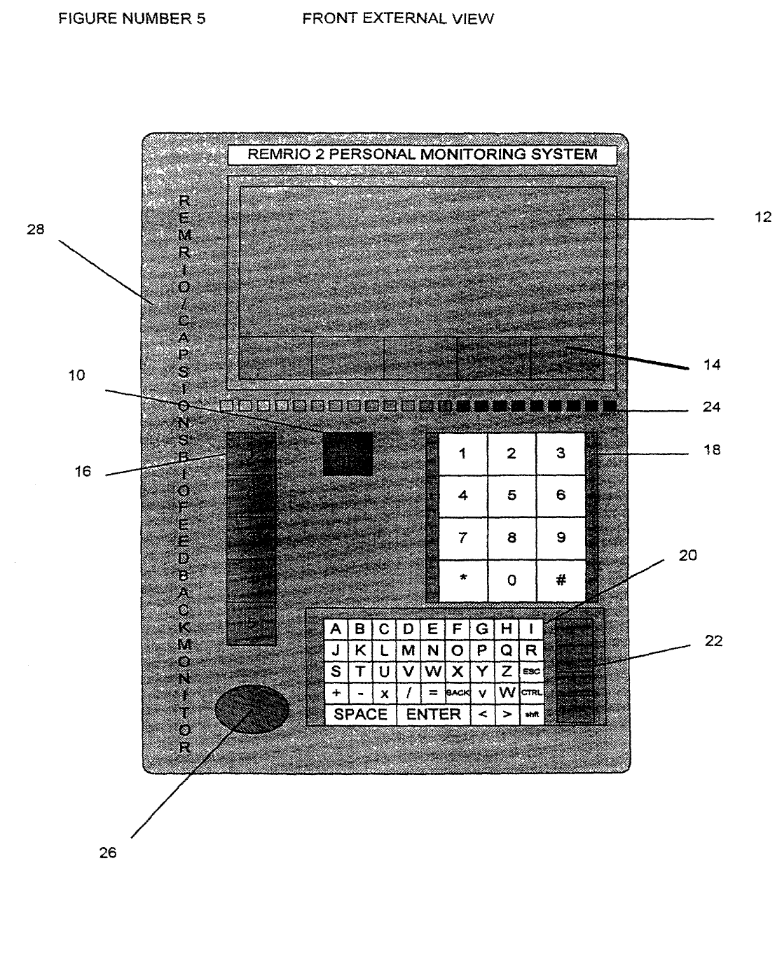

### Operation—FIGS. 1 to 7

The manner of operation and usage of the RT/BIOCAPSIONS Biofeedback Monitor by consumers, clinicians, industrial and radiation workers, military personnel, and skin tanners for real-time monitoring and measurement of biological and/or physical and/or chemical sensitivity, irritation and injury onset is shown in FIGS. 1 to 7. The portable biofeedback monitor and applications system; the size of a Walkman radio/CD player, as well as smaller versions the size of a large watch, pager or cell phone can be clipped onto or concealed underneath the clothing of chemically sensitive individuals. Once the monitor is calibrated to the user(test article) by challenge testing. The user can accurately monitor the onset of biological and/or chemical and/or physical sensitivity and irritation by selecting a pre-programmed parameter function key. The monitor generates a prognostic out using the device's visual, audible and vibratory alerts.

From the description above, a number of advantages of my REMRIO/BIOCAPSIONS invention become evident: A device for determination of allergy sensitivity onset, asthma sensitivity onset, multiple chemical sensitivity onset, physical sensitivity onset, time weighted averages, occupational threshold limit values, radiation injury onset, sunburn injury onset, neurogenic inflammation onset. A device for determining spontaneous discharge patterns, evoked potentials, conduction velocities, pharmacological modulation and automatic biofeedback evoked transdermal delivery of protective agents for advanced military personnel protection. A device for determination and expression of Human BIOCAPSIONS receptive fields and biosensors, analog feature node expression, pattern constant processing, pattern-directed retrieval, connectivity, matching, recognition, variability, classification, pattern-directed modulation, pattern-directed interfacing, stimulus-equivalence reconciliation, bio-feature detection and association and any and all embodiments within the scope of the invention.

Although the description above contains many specifications, these should not be construed as limiting the scope of the invention but as merely providing illustrations of some of the presently preferred embodiments of this invention. For example, the present housing can be replaced by smaller housing similar to a pager, cell phone or electrode jacks can be replaced with spliced and/or multi port jacks to accommodate the use of more than two or three electrode, etc. or remote programming and data downloading.

From the foregoing description, one skilled in the art can easily ascertain the essential characteristics of this invention and, without departing from the spirit and scope thereof, can make various changes and modifications of the invention to adapt it to various usages and conditions. For example,

Thus the scope of the invention should be determined by the apprehended claims and their legal equivalents, rather than by the examples given. It is to be understood that the present invention is not limited to the sole embodiment described above, but encompasses any and all embodiments within the scope of the following claims:

I claim

1. A method using a computer processor to measure internal environment of the body by analyzing neuronal-based electrical signals. A device for preventing over-exposure to biological, chemical and physical hazards. More specifically, the device and processor performs analog feature node expression, pattern constant processing and stimulus-equivalence reconciliation.

2. The device records, analyzes and displays data representative of the human neuro-sensory activities of thermo, chemo, mechano, photo and gravi receptor potentials.

3.The computer processor makes approximations of biosensor receptive fields, connectivity, matching, recognition, variability, and neurodynamics classification.

4. Additionally, other neurodynamics data are analogically bio-featured, detected and associated with predetermined thresholds.

5.The artificial intelligence scope of device records and approximates specificity, intensity, gradation, adaptation, amplification, and transduction plasticity, convergence, divergence, and circuits and pathways.

6. The computer processor performs pattern-directed modulation, pattern-directed retrieval, and pattern-directed interfacing.

7. The computer processor makes approximations of electrotonic potentials, impulse discharge coding, periods and frequency, and signaling and propagation in response to variable intensity biological, physical, and chemical stimulation.

8. The device and apparatus is a dual analog monitoring and digital measurement device where the embodiment includes a stimulating, two recording and transdermal pharmacological delivery electrodes. Other embodiments include a differential amplifier, A/D converter, digital filtering; digital signal processor, software, and any and all embodiments within the scope of the invention.

9. In military and industrial hygiene support, this low-powered, miniaturized and field portable measurement device can be used to timely capture and distribute clinical information using network technology by utilizing the device's diagnostic capability, continuous data acquisition, non-invasive sensing of vital signs and body chemistry, immediacy of analysis prior to injury or illness, ability to transmit this information to a distance monitoring facility to support decisions to plan and manage overall responses.

10. measurement device for transdermal delivery of protective agents for advanced military personnel protection and chronopharmacological intervention and modulation.

11. The measurement device detects nerve impulse propagation and make a determination of industrial hygiene time weighted averages and threshold limit values.

12. The measurement device detects nerve impulse propagation and make a determination of sensitivity, irritation and injury onset for allergens, asthmatics, biological, chemical and physical sensitizers.

13. The measurement device detects nerve impulse propagation and make a determination of sunburn, ionizing radiation, and nonionizing injury onset.

14. The measurement device detects nerve impulse propagation and make a determination of human neurogenic inflammation onset.

15. The measurement device's second recording electrode give the device the capability to measure multi-modal physiological parameters: such as EMG, EEG, temperature, heart rate, blood volume pulse, skin conductance, skin perspiration, EKG, and respiration; integrate said auxiliary physiological measurements into CAPSIONS impulse pattern recognition; and display said physiological measurements on the device's real-time display screen.

16. The measurement device's fourth recording electrode give the device the capability detect and measure noradrenic and serotonogenic specific impulse propagation; integrate said auxiliary physiological measurements into BIOCAPSIONS impulse pattern recognition; perform chronopharmacological modulation; and display said physiological modulated measurements on the device's real-time display screen.

17. The measurement device can be used as a neuronal-based user-machine calibration standard for similar devices; by creating a base-line reference correlating to chemical and physical stimulation challenge; store said calibration data in the device's memory; and display said calibration on the device's display screen.
